(19) United States
(12) Patent Application Publication
(10) Pub. No.: US 2008/0312154 A1
(43) Pub. Date: Dec. 18, 2008

(54) ADMINISTRATION OF ADIPOCYTOKINE AND ADIPONECTIN FOR TREATING HEADACHE AND METHODS OF DIAGNOSING HEADACHE BY MEASURING ADIPONECTIN

(76) Inventor: Barbara Lee Peterlin, Philadelphia, PA (US)

Correspondence Address: PIETRAGALLO GORDON ALFANO BOSICK & RASPANTI LLP, ONE OXFORD CENTRE, 38TH FLOOR, 301 GRANT STREET, PITTSBURGH, PA, 15219-6404, US

(21) Appl. No.: 12/138,812

(22) Filed: Jun. 13, 2008

Related U.S. Application Data
(60) Provisional application No. 60/934,690, filed on Jun. 15, 2007.
(60) Provisional application No. 60/981,852, filed on Oct. 23, 2007.

(51) Int. Cl. A61K 38/17; C07K 14/575; C12Q 1/00

(52) U.S. Cl. 514/12; 530/399; 435/4

(57) ABSTRACT

Methods for treating a headache, such as a migraine, in a subject comprising administering a therapeutically effective amount of an adipocytokine, for example, adiponectin, to the subject are disclosed. Pharmaceutical compositions comprising adipocytokine, for example, adiponectin, are disclosed. Methods of diagnosing the presence or absence of a headache in a subject, such as a migraine, by assessing the level of adiponectin in the subject are disclosed.

### CROSS REFERENCE TO RELATED APPLICATIONS

This application claims benefit of U.S. Provisional Application No. 60/934,690 filed Jun. 15, 2007, and U.S. Provisional Application No. 60/981,852 filed Oct. 23, 2007.

The entire teachings of the above applications are incorporated herein by reference.

### FIELD OF THE INVENTION

The present invention is generally in the field of administration of an adipocytokine, for example adiponectin, to a subject to treat a headache, such as a migraine, and to pharmaceutical compositions comprising adipocytokine, for example adiponectin, as well as to diagnosing a headache, such as a migraine, in a subject, including by determining the level of adiponectin in a subject.

### BACKGROUND INFORMATION

Headache is a common type of pain suffered by many. Among the different types of headache is a migraine. Migraine is the most common neurological condition in the developed world. It affects 10% of the U.S. population and is more prevalent than diabetes, epilepsy and asthma combined. Migraine is more than just a headache. It can be a debilitating condition which has a considerable impact on the quality of life of sufferers and their families. Migraineurs come from all walks of life, all areas of the world and ethnic groups, and all social classes. Attacks can be completely disabling, forcing the sufferer to abandon everyday activities for up to 3 days. Even in symptom-free periods, sufferers may live in fear of the next attack. Migraine attacks normally last between 4 and 72 hours and sufferers are usually symptom free between attacks. Migraine is a complex condition and a treatment which is successful for one patient may have no effect on another.

It would therefore be desirable to develop a method of diagnosing the presence or absence of a headache, such as a migraine, in a subject, as well as to treat a subject with a headache, such as a migraine, by administering a pharmaceutical composition.

The present invention has been developed in view of the foregoing.

### SUMMARY OF THE INVENTION

One aspect of the present invention relates to a method for treating a headache in a subject comprising administering a therapeutically effective amount of an adipocytokine to the subject.

Another aspect relates to a pharmaceutical composition comprising adipocytokine and a pharmaceutically acceptable carrier for administration of a therapeutically effective amount of the adipocytokine to treat a subject with a headache.

Yet another aspect of the present invention relates to use of adipocytokine for manufacture of a medicament for treatment of a headache.

Still another aspect of the present invention relates to a method of diagnosing the presence or absence of a headache in a subject, the method comprising: assessing a test sample from the subject for a level of adiponectin, wherein the presence of a level of adiponectin that is greater than a level of adiponectin in a comparable negative control sample, by an amount that is statistically significant, is indicative of the presence of headache in the subject, and wherein the presence of a level of adiponectin that is equal to or less than a level of adiponectin in a comparable negative control sample, by an amount this is statistically significant, is indicative of the absence of headache in the subject.

These and other aspects will become more apparent from the following description.

### DETAILED DESCRIPTION

Other than in the operating examples, or where otherwise indicated, all numbers expressing quantities of ingredients, reaction conditions and so forth used in the specification and claims are to be understood as being modified in all instances by the term “about”. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical values, however, inherently contain certain errors necessarily resulting from the standard deviation found in their respective testing measurements.

Also, it should be understood that any numerical range recited herein is intended to include all sub-ranges subsumed therein. For example, a range of “1 to 10” is intended to include all sub-ranges between and including the recited minimum value of 1 and the recited maximum value of 10, that is, having a minimum value equal to or greater than 1 and a maximum value of equal to or less than 10.

In this application, the use of singular includes the plural and plural encompasses singular, unless specifically stated otherwise. In addition, in this application, the use of “or” means “and/or” unless specifically stated otherwise, even though “and/or” may be used in certain instances.

Adipocytokines are a group of cytokines (cell-to-cell signaling proteins) secreted by adipose tissue. Adipose tissue produces chemokines and cytokines, as well as adipocytokines. Adipocytokines are mainly adipocyte-derived cytokines regulating metabolism and are key regulators of insulin resistance. Adipocytokines include adiponectin, leptin, resistin, visfatin, adipsin, and the like.

Adiponectin (ADP) is an adipocytokine that plays a role in energy homeostasis, has protective roles against the development of insulin resistance and atherosclerosis, and exhibits both pro-inflammatory and anti-inflammatory properties. Adiponectin (ADP) is a protein that is a member of the adipocytokines family and is primarily secreted from adipocytes in adipose tissue, although it is also secreted in low levels from cardiomyocytes, hepatocytes, and the placenta. It exhibits a sexual dimorphism, with females having higher levels than males by puberty. Human plasma ADP can exist as a full-length form, a smaller fragment of the full length form (which is formed by cleavage of full length ADP by proteases) termed globular adiponectin (gADP) or as one of several characteristic oligomers or multimers, including high molecular weight adiponectin (HMW-ADP), middle molecular weight adiponectin (MMW-ADP), or low molecular weight adiponectin (LMW-ADP). LMW-ADP is a trimer formed via hydrophobic interactions within its globular domain. Two trimers form a disulfide-linked hexamer or MMW-ADP, which assembles into a multimeric complex of 12-18 monomers or HMW-ADP.

The ability of ADP to exert both pro-inflammatory and anti-inflammatory properties appears to be determined by the form of ADP involved. Human globular ADP (gADP) activates the pro-inflammatory NFkβ pathways, as well as induces the secretion of the pro-inflammatory cytokines (IL-6 and TNF-α). The pro-inflammatory NFkβ pathways, IL-6, and TNF have all been shown to be increased during acute migraine attacks. However, under certain conditions, gADP exerts anti-inflammatory properties. In addition, the different multimers of ADP have been shown to activate different pathways and have distinct functions. Of the multimers of ADP, HMW-ADP is the only one that has been shown to activate the pro-inflammatory NFkβ pathways in humans. While HMW-ADP has been shown to induce the secretion of IL-6, LMW-ADP has been shown to reduce IL-6 secretion. In migraine, serum levels of IL-6 have been shown to be elevated during acute attacks, and adiponectin and its multimers, HMW-ADP and MMW-ADP, have been shown to be elevated in chronic daily headache sufferers with either chronic migraine or medication overuse headache.

In certain embodiments, the present invention provides methods for treating a headache in a subject comprising administering a therapeutically effective amount of an adipocytokine to the subject. In other embodiments, the headache is a migraine. In certain embodiments, the adipocytokine comprises adiponectin.

As used herein, the term “subject” includes animals, such as mammals, including, but not limited to, primates (e.g., humans), cows, sheep, goats, horses, dogs, cats, rabbits, rats, mice, and the like.

As used herein, the terms “treating” or “treatment” of a disease, for example, a headache and/or a migraine, includes: (1) preventing the disease, i.e., causing the clinical symptoms of the disease not to develop in a subject that may be exposed to or predisposed to the disease but does not yet experience or display symptoms of the disease, (2) inhibiting the disease, i.e., arresting or reducing the development of the disease or its clinical symptoms, or (3) relieving the disease, i.e., causing regression of the disease or its clinical symptoms.

As used herein, the term “therapeutically effective amount” means the amount of the subject compound that will elicit the biological or medical response of a tissue, system, animal or human that is being sought by the researcher, veterinarian, medical doctor or other clinician. A “therapeutically effective amount” includes the amount of a compound that, when administered to a mammal for treating a disease, is sufficient to effect such treatment for the disease. The “therapeutically effective amount” will vary depending on the compound, the disease and its severity and the age, weight, etc., of the mammal to be treated. The amount which will be therapeutically effective will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms, and should be decided according to the judgment of a practitioner and each subject's circumstances. Effective doses may be extrapolated from dose-response curves derived from in vitro or animal model test systems.

In certain embodiments, the present invention provides a pharmaceutical composition comprising adipocytokine and a pharmaceutically acceptable carrier or excipient for administration of a therapeutically effective amount of the adipocytokine to treat a subject with a headache. In certain embodiments, the headache is a migraine. In other embodiments, the adipocytokine comprises adiponectin.

As used herein, the term “adiponectin” includes full length adiponectin; fragments of adiponectin, for example, a globular head of adiponectin; adiponectin as a monomer, oligomer, or as a multimer (two or more adiponectin molecules attached or bound to, or otherwise interacting with, one another); an adiponectin receptor agonist; an adiponectin receptor antagonist; nucleic acids encoding adiponectin or the globular domain of adiponectin, and vectors comprising such nucleic acids (e.g., a gene, cDNA, and/or mRNA); agents that enhance or increase interaction between adiponectin and an adiponectin binding agent (e.g., an agent that enhances or increases interaction between adiponectin and its receptor); agents that enhance or increase activity of an adiponectin receptor; agents that increase activity of proteins that influence release of adiponectin (e.g., agonists of peroxisome proliferator-activated receptor gamma (PPAR-gamma)); agents that stimulate release or secretion of adiponectin; and adiponectin therapeutic agents. As used herein, the term “adiponectin therapeutic agent” includes any agent that enhances adiponectin activity. Adiponectin therapeutic agents can alter adiponectin activity by a variety of means, such as, for example, by providing additional adiponectin; by upregulating the transcription or translation of the adiponectin gene; by upregulating or increasing the release of adiponectin; by altering posttranslational processing of adiponectin; by altering the interaction between adiponectin and an adiponectin binding agent (e.g., a receptor); by altering the activity of an adiponectin binding agent (e.g., enhancing activity of a receptor). Some of the aforementioned materials may fall into more than one category.

In certain embodiments, the adiponectin comprises natural adiponectin, synthetic adiponectin, full length adiponectin, fragments of adiponectin, monomeric adiponectin, multimeric adiponectin, adiponectin agonists, adiponectin antagonists, or combinations thereof.

Adiponectin agonists or antagonists may include any that are known in the art, but in certain embodiments, are those related to human adiponectin such as those that may be derived by molecular biology (including recombinant techniques), microbiology, cell biology, biochemistry, nucleic acid chemistry, and immunology techniques.

In certain embodiments, the fragments of adiponectin comprise globular adiponectin, for example a globular head of adiponectin. In still other embodiments, the multimeric adiponectin comprises high molecular weight adiponectin (HMW-ADP), middle molecular weight adiponectin (MMW-ADP), low molecular weight adiponectin (LMW-ADP), or combinations thereof, such that both monomeric adiponectin and multimeric adiponectin can be used concurrently. In other embodiments, the adiponectin may be modified in some way, including but not limited to, for example, glycosylated.

In certain embodiments, any combination of one or more types of adiponectin, such as any of those previously mentioned, may be used.

As discussed above, in certain embodiments, the methods of the present invention comprise administering a therapeutically effective amount of adipocytokine to a subject to treat a headache. In other embodiments, the adipocytokine comprises adiponectin. In certain embodiments, the therapeutically effective amount of adiponectin or other adipocytokines comprises a total daily dosage amount of 0.01 μg/ml to 100 μg/ml, such as from 1 μg/ml to 25 μg/ml, wherein “μg/ml” means micrograms per milliliter. In certain embodiments, the total daily dosage amount may be administered to a subject once a day. In other embodiments, the total daily dosage amount may be administered to a subject more than once a day.

In certain embodiments of the present invention, the adiponectin or other adipocytokines may be administered alone, while in other embodiments, one or more other additional active ingredients may be used in combination with the adiponectin or other adipocytokines. In certain embodiments, these additional active ingredients may have related utilities to prevent and/or treat headache and/or migraine, as well as augment the effectiveness of the adiponectin or other adipocytokines in alleviating or ameliorating a headache and/or migraine. In other embodiments, these additional active ingredients may have effectiveness to prevent and/or treat any side effects associated with a headache and/or migraine, including but not limited to, for example, nausea.

In certain embodiments, the additional active ingredients may be administered to a subject, by a route and in an amount commonly used therefore, contemporaneously or sequentially with adiponectin or other adipocytokines. In other embodiments, these additional active ingredients may be present in a pharmaceutical composition in combination with adiponectin or other adipocytokines, for example, when adiponectin or other adipocytokines is used contemporaneously with one or more additional active ingredients.

In certain embodiments, adipocytokines other than adiponectin may used in combination with adiponectin in the methods and pharmaceutical compositions of the present invention. Non-limiting examples of other adipocytokines include leptin, resistin, visfatin, adipsin, and the like. Any combination of one or more of the aforementioned may also be used.

In certain embodiments of the present invention further comprises co-administering a therapeutically effective amount of an analgesic comprising an opioid analgesic, a non-opioid analgesic, or combinations thereof; non-steroidal anti-inflammatory agents; orexin modulating agents, including, for example, orexin receptor antagonists, and the like; dopamine modulation agents, including, for example, metoclopramide, prochlorpromaxine, and the like; serotonin modulating agents, including, for example, triptans, SNRI compounds (Serotonin Norepinephine Reuptake Inhibitors), SSRI compounds (Selective Serotonin Reuptake Inhibitors), and the like; or any combination of one or more of any of the above-mentioned compounds, along with the administration of a therapeutically effective amount of adiponectin or other adipocytokines to treat a subject with a headache, such as a migraine. These additional active ingredients may be administered, by a route and in an amount commonly used therefore, contemporaneously or sequentially with the adiponectin or other adipocytokines. In other embodiments, one or more of these additional active ingredients may be present in a pharmaceutical composition in combination with the adiponectin or other adipocytokines.

In certain embodiments, analgesics comprising opioid analgesics, non-opioid analgesics, or combinations thereof, may be administered to a subject as additional active ingredients in combination with adiponectin or other adipocytokines, while in other embodiments, these analgesics may be present as additional active ingredients in a pharmaceutical composition.

In certain embodiments, the non-opioid analgesic comprises a non-steroidal anti-inflammatory agent. Non-limiting examples of NSAIDS include, for example, propionic acid derivatives (e.g., alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, tioxaprofen, and the like); acetic acid derivatives (e.g., indomethacin, acemetacin, aldlofenac, clidanac, diclofenac, fenclofenac, fenclozic acid, fentiazac, furofenac, ibufenac, isoxepac, oxpinac, sulindac, tiopinac, tolmetin, zidometacin, zomepirac, and the like); fenamic acid derivatives (e.g., flufenamic acid, meclofenamic acid, mefenamic acid, niflumic acid, tolfenamic acid, and the like); biphenylcarboxylic acid derivatives (e.g., diflunisal, flufenisal, and the like), oxicams (isoxicam, piroxicam, sudoxicam, tenoxican, and the like); salicylates (e.g., acetyl salicylic acid, sulfasalazine, and the like); pyrazolones (apazone, bezpiperylon, feprazone, mofebutazone, oxyphenbutazone, phenylbutazone, and the like); indomethacin; acetaminophen; and salicylic acid derivatives (aspirin, diclofenac, ketorolac, piroxicam, meloxicam, mefenamic acid, sulindac, tolmetin sodium, zomepirac, fenoprofen, phenylbutazone, oxyphenbutazone, nimesulide, zaltoprofen, letodolac, and the like). Any combination of one or more of the aforementioned NSAIDS may also be used in the methods and pharmaceutical compositions of the present invention.

In other embodiments, the analgesic may comprise an opioid analgesic. Non-limiting examples of suitable opioid analgesics include alfentanil, apomorphine, buprenorphine, butorphanol, codeine, dextropropoxyphene, diamorphine, dihydrocodeine, fentanyl, hydrocodone, hydromorphone, levorphanol, meperidine, meptazinol, methadone, morphine, nalbuphine, oxycodone, oxymorphone, pentazocine, propoxyphene, sufentanil, tramadol, and the like, or combinations thereof. Any combination of one or more opioid analgesics may be used.

In certain embodiments, an orexin modulating agent may be administered in combination with or be present in a pharmaceutical composition in combination with the adiponectin or other adipocytokines. Any orexin modulating agent known in the art may be used, including, for example, orexin receptor antagonists. Any combination of one or more orexin modulating agents may be used.

In certain embodiments, a dopamine modulating agent may be administered in combination with the adiponectin or other adipocytokines. In other embodiments, a dopamine modulating agent may be present in a pharmaceutical composition in combination with the adiponectin or other adipocytokines. Any dopamine modulating agent known in the art may be used, including, for example, metoclopramide, prochlorpromaxine, and the like. Any combination of one or more dopamine modulating agents may be used.

In certain embodiments, a serotonin modulating agent may be administered in combination with the adiponectin or other adipocytokines. In other embodiments, a serotonin modulating agent may be present in a pharmaceutical composition in combination with the adiponectin or other adipocytokines. Any serotonin modulating agent known in the art may be used, including, for example, triptans, SNRI compounds, SSRI compounds, and the like. Any combination of one or more serotonin modulating agents may be used.

Besides those additional active ingredients discussed above, in certain embodiments, other ingredients may be administered in combination with or may be present in a pharmaceutical composition in combination with adiponectin or other adipocytokines. Non-limiting examples of these ingredients include, for example, anti-nausea agents. Any anti-nausea agent known in the art may be used, including, for example, metoclopramide, prochlorperazine, odansetrom, and the like. Any combination of one or more anti-nausea agents may be used.

As discussed above, any combination of any additional active ingredients may be used. If any additional active ingredients are present, the weight ratio of adiponectin or other adipocytokines to these ingredients may be varied and will depend upon the effective dose of each ingredient. Generally, a therapeutically effective dose of each will be used.

As discussed above, in certain embodiments the adipocytokine may be present in a pharmaceutical composition along with a pharmaceutically acceptable carrier or excipient for administration of a therapeutically effective amount of the adipocytokine to treat a subject with a headache. In certain embodiments, the headache is a migraine. In other embodiments, the adipocytokine comprises adiponectin.

As used herein the term “pharmaceutical composition” includes any appropriate composition well known in the art to be suitable for pharmaceutical formulation of proteins suitable for administration to a subject, for example to mammals, particularly (although not solely) those suitable for stabilization in solution of therapeutic proteins for administration to mammals, irrespective of whether or not the adiponectin is in the form of a composition. In certain embodiments, the pharmaceutical composition may be an immediate release formulation, while in other embodiments, it may be a controlled or sustained release formulation.

As used herein, the term “pharmaceutically acceptable carrier or excipient” means a carrier or excipient that is useful in preparing a pharmaceutical composition that is generally safe, non-toxic and neither biologically nor otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. The carrier and composition can be sterile. The formulation should suit the mode of administration.

Suitable pharmaceutically acceptable carriers or excipients include, but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., or combinations thereof. The pharmaceutical compositions can, if desired, be mixed with auxiliary agents, e.g., lubricants, preservatives, stabilizers, wetting agents, emulsifiers, salts for influencing osmotic pressure, buffers, coloring, flavoring and/or aromatic substances, and the like, which do not deleteriously react with the active agents in the compositions. Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. The compositions, if desired, may also contain minor amounts of wetting or emulsifying agents, or pH buffering agents, and the like.

In certain embodiments, the adiponectin or other adipocytokines is present in the pharmaceutical composition at a total amount of 0.01 μg/ml to 100 μg/ml, such as from 1 μg/ml to 50 μg/ml, such as from 5 μg/ml to 25 μg/ml, wherein “μg/ml” means micrograms per milliliter. In certain embodiments, the pharmaceutical composition may be administered to a subject once a day, while in other embodiments, the pharmaceutical composition may be administered to a subject more than once a day. In certain embodiments, the pharmaceutical composition may be formulated as an immediate release formulation while in other embodiments, the pharmaceutical composition may be a controlled or sustained release formulation.

In certain embodiments, the pharmaceutical compositions may be prepared for oral, enteral, parenteral, pulmonary, nasal, subcutaneous, intramuscular, intravenous, transdermal, topical, suppository, transmucosal, subdermal delivery, or combinations thereof. The pharmaceutical compositions can be a liquid solution, suspension, emulsion, tablet, pill, capsule, controlled or sustained release formulation, or powder.

The pharmaceutical compositions can be formulated in accordance with the routine procedures as a pharmaceutical composition adapted for administration to, for example, human beings.

Non-limiting examples of oral delivery include tablets, capsules, lozenges, and the like, or any liquid forms such as syrups, aqueous solutions, emulsion, and the like, capable of protecting the therapeutic protein from degradation prior to eliciting an effect, e.g., in the alimentary canal if an oral dosage form. Oral formulations can include standard carriers such as pharmaceutical grades of mannitol, lactose, starch, magnesium stearate, polyvinyl pyrollidone, sodium saccharine, cellulose, magnesium carbonate, and the like.

Non-limiting examples of transdermal delivery including transdermal patches, transdermal bandages, and the like. Non-limiting examples of topical delivery include any lotion, stick, spray, ointment, paste, cream, gel, etc. whether applied directly to the skin or via an intermediary such as a pad, patch, and the like. For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Non-limiting examples of suppository delivery include any solid or other dosage form to be inserted into a bodily orifice (particularly those inserted rectally, vaginally and urethrally). Non-limiting examples of transmucosal delivery includes depositories, solutions for enemas, pessaries, tampons, creams, gels, pastes, foams, nebulised solutions, powders and similar formulations containing in addition to the active ingredients such carriers as are known in the art to be appropriate. Non-limiting examples of depot administration includes pellets or small cylinders of active agent or solid forms wherein the active agent is entrapped in a matrix of biodegradable polymers, microemulsions, liposomes or is microencapsulated. Non-limiting examples of delivery via bolus include single or multiple administrations by intravenous injection, subcutaneous, subdermal, and intramuscular administration or oral administration. Non-limiting examples of pulmonary/nasal delivery include inhalation or insufflation include compositions comprising solutions and/or suspensions in pharmaceutically acceptable, aqueous, or organic solvents, or mixture thereof and/or powders.

In certain embodiments, the present invention includes use of adipocytokine for the manufacture of a medicament for the treatment of a headache, including for example, a migraine. In certain embodiments, the adipocytokine comprises adiponectin.

In certain embodiments, the present invention relates to a method of diagnosing the presence or absence of a headache in an individual, the method comprising: assessing a test sample from the individual for the level of adiponectin, wherein the presence of a level of adiponectin that is greater than a level of adiponectin in a comparable negative control sample, by an amount that is statistically significant, is indicative of the presence of headache in the individual, and wherein the presence of a level of adiponectin that is equal to or less than a level of adiponectin in a comparable negative control sample, by an amount this is statistically significant, is indicative of the absence of headache in the individual. In other embodiments, the headache is a migraine.

In the methods of the invention, a “test sample” from a subject to be assessed for a headache and/or migraine, or for risk of a headache and/or migraine is used. The test sample can comprise blood, serum, cerebrospinal fluid, urine, nasal secretion, saliva, or any other bodily fluid or tissue. In one embodiment, the test sample is a blood or serum sample from the subject. In another embodiment, the subject to be assessed for a headache and/or migraine or for risk of headache and/or migraine is a woman. The level of adiponectin in the test sample is then measured, using standard methods, such as by enzyme-linked immunosorbent assay (ELISA).

As used herein, the terms “statistically significant,” “statistically significant difference,” and the like have the normal meaning in the art and means that the probability of the observed difference (or in the case of “statistically similar” measurements, the probability of a observed absence of difference) occurring by chance (the p-value) is less than some predetermined level, i.e., a p-value that is <0.05, preferably <0.01 and most preferably <0.001. A variety of suitable statistical methods are well known to those skilled in the art can be used to measure statistical significance (e.g., standard statistical methods such as Student t-tests (for comparing two samples), ANOVA (analysis of variance), and confidence interval analysis; software such as the SAS System Version 8 (SAS Institute Inc., Cary, N.C., USA) can be used for analysis.

In certain embodiments of the invention, the test sample is assayed to determine the level of adiponectin, as above. The level of adiponectin in the test sample is compared with the level of adiponectin in at least one comparable negative control sample (i.e., a sample from an individual who is not affected by headaches and/or migraine). The negative control sample can be a sample from any individual who is not affected by headaches and/or migraine; it is not necessary that the negative control sample be from an individual who is free of disease. A “comparable” negative control sample is a sample of the same type of body fluid or tissue as the test sample. More than one control sample can be used.

In these embodiments, the presence of a level of adiponectin in the test sample that is significantly greater than the level of adiponectin in a comparable control sample(s), as described above, correlates with the presence of headache and/or migraine and/or a risk of headache and/or migraine. The presence of a level of adiponectin in the test sample that is equal to or less than the level of adiponectin in a comparable control sample(s), by an amount this is statistically significant, is correlates with an absence of headache and/or migraine and/or a risk of headache and/or migraine in a subject.

The methods of diagnosis described above can be applied in a similar manner to assess a subject for a risk of relapse after treatment for a headache and/or migraine. A “risk of relapse,” as used herein, refers to an adiponectin-associated risk for the return of the headache and/or migraine after treatment. While other risk factors may exist for relapse, the methods described herein pertain to risk associated with levels of adiponectin.

In other embodiments of the invention, the methods described above with regard to headache and/or migraine can be applied in a similar manner to other types of neurological pain.

The following Examples are offered for the purpose of illustrating the present invention and are not to be construed to limit the scope of the invention.

### EXAMPLES

### Correlation Between Adiponectin Levels and Headache

Obesity is a risk factor for headache chronification. Adiponectin (ADP) is an adipocytokine secreted primarily by adipose tissue. ADP and its oligomers (HMW, MMW and LMW adiponectin) have been shown to modulate several inflammatory pathways which have been shown to be associated with migraine pathophysiology.

Age and body mass index (BMI) matched female participants were enrolled. Anthropometric measures (including waist-to-hip ratio (WHR) and BMI)) were measured in all participants. Serum total adiponectin levels and its oligomers were measured in EM during headache-free periods and CDH sufferers at baseline level of pain, as compared to healthy control subjects using enzyme-linked immunoabsorbent assays (ELISA).

Results: Although total body obesity as estimated by BMI, showed no significant association between participants, visceral obesity as estimated by WHR was significantly associated with chronic daily headache suffers (CDH) as compared to episodic migraineurs (EM) and controls. WHR was also significantly and inversely related to both total-ADP (T-ADP), (p=0.008) and high molecular weight ADP (HMW-ADP), (p=0.002). After adjusting for WHR, serum T-ADP levels were significantly higher in CDH sufferers (10.1±4.0) than in both EM (8.6±3.5) and controls (7.5±2.4), p=0.024. In addition high molecular weight ADP (HMW-ADP) was higher in CDH (6.1±2.8) as compared to EM (4.2±1.7) and controls (3.9±1.5), p=0.003. And middle molecular weight ADP (MMW-ADP) was also higher in CDH (2.0±1.2) as compared to EM (1.5±0.7) and controls (1.1±0.4), p=0.009.

Conclusion: Serum adiponectin levels are elevated in female CDH sufferers. In addition visceral obesity is a stronger risk factor for CDH than EM.

### Methods

### Patients and Controls

A total of 37 age and body mass index (BMI) matched, normotensive, nondiabetic, caucasian female participants were included in the study. Headache diagnoses were classified according to the Second Edition of the International Classification of Headache Disorders (ICHD-2). According to their diagnoses, migraine participants were divided into two groups. Group one consisted of 12 chronic daily headache (CDH) patients having an ICHD-2 code of probable medication over-use headaches (MOH) or chronic migraine (CM). Group two consisted of 13 episodic migraineurs (EM) with an ICHD-2 code of migraine with aura (MA) or migraine without aura (MO). The control group consisted of 12 healthy female blood donors. Participants with a physician diagnosis of diabetes, coronary artery disease, thyroid disease, hypercholesterolemia, recent infection or renal disease were excluded. Characteristics of the groups are presented in Table 1.

Serum blood samples were collected interictally in episodic migraineurs, at baseline level of pain in chronic daily headache sufferers and pain-free in control subjects between 9 am and 4 pm. Informed, written consent was obtained from all participants.

### Anthropometric Measurements

In general it is accepted that the regional distribution of adipose tissue, and not only total body fat, is associated with the development of several diseases including hypertension, coronary heart disease, diabetes and stroke. In particular, visceral or abdominal obesity, as estimated by a high waist circumference (WC) or high waist-to-hip ratio (WHR), independent of total body obesity, (as estimated by body mass index (BMI)) have been shown to be better predictors for disease than total body obesity estimates, i.e. BMI. Thus, the anthropometric measurements for both total body obesity (BMI) and abdominal obesity (WC and WHR) were calculated for all participants.

Measurements of Total Body Obesity: Height was measured to the nearest 0.5 inch with a mounted stadiometer. Weight was measured with a standard scale to the nearest 0.5 lb. BMI was then calculated using the formula: BMI=wt [lbs]/ht²*703, and categorized on the basis of the World Health Organization (WHO) categories: <18.5 (underweight), 18.5 to 24.9 (normal weight), 25 to 29.9 (overweight) and ≧30 kg/m² (obese).

Measurements of Regional Obesity: Waist and hip circumferences were measured (cm) with an anthropometric tape over skin or light clothing. Waist circumference (WC) was measured at the minimum circumference between the iliac crest and the rib cage. Hip circumference (HC) was measured at the maximum width over the greater trochanters. For the definition of abdominal obesity based on WC, 88 cm was used according to the recommendations for the definition of metabolic syndrome, corresponding to a BMI of 30. Additionally a WC of 80-88 cm was used to identify individuals at increased cardiovascular risk. The WHR was calculated by dividing the WC by the HC and categorized based on the world health organization recommendations of a WHR greater than 0.85 being considered increased in women.

### Laboratory Methods

After sampling in EDTA or serum tubes, blood was immediately chilled on ice, then centrifuged, aliquoted and stored at a temperature of −70° C. until assayed.

Glucose: Glucose levels were evaluated clorimetricaly in 96 well plates with the QuantiChrom glucose assay kit (BioAssay Systems, Hayward, Calif.). Absorbance was determined with a Spectramax 190 spectrophotometer (Molecular Devices, Sunnyvale, Calif.)

Adiponectin: Serum levels of total adiponectin, HMW adiponectin and combined HMW+MMW adiponectin were determined by multimeric enzyme immunosorbent assay (ALPCO, Salem, N.H.) The MMW-ADP concentration was calculated by subtracting the HMW-ADP concentration from the combined MMW+HMW adiponectin concentration. The LMW-ADP concentration was calculated by subtracting the combined HMW+MMW adiponectin concentration from the total adiponectin concentration. All assays were performed in duplicate according to the manufacturers' instructions. Recent data has suggested that HMW-ADP may be more relevant in the prediction of insulin resistance and that the ratio of HMW-ADP to T-ADP may be a more significant predictor of disease than T-ADP. Thus, the ratio of HMW-ADP to T-ADP (HMWR) was calculated for all participants.

### Statistical Analysis

Statistical analyses were carried out using SPSS, version 15 (SPSS Inc, Chicago, Ill.). All data are presented as mean ±SD where appropriate. For demographic variables analysis of variance or independent Sample T-tests were calculated for quantitative variables. Nominal and categorical data were analyzed by chi square. For outcome variables analysis of variance was calculated for T-ADP, its isomers and the HMWR for each group. Bivariate correlations were done to test relationships between controls and outcome variables. Linear multiple regression analyses were conducted to model the strength of various measures as correlates of T-ADP, its isomers, and the HMWR including the WHR and serum glucose.

### Results

All patients and controls completed the study. The concentration of adiponectin was above the detection level in all blood samples.

The demographic characteristics and serum glucose levels showed no significant difference between CDH sufferers, EM and controls (See Table 1). BMI and WC were not significantly associated with serum adiponectin levels or any of the ADP isomers.

However, WHR was significantly correlated with both T-ADP, (p=0.008) and HMW-ADP, (p=0.002) (See Table 2). Mean estimates of total body fat (BMI) and mean abdominal obesity (WHR) were not significant between groups. In addition, based on the WHO recommendations, the percentage of participants with a BMI consistent with obesity (i.e., BMI≧30) was not significant between groups. However, the percentage of CDH sufferers with abdominal obesity (i.e., WHR≧0.85) was significantly greater as compared to EM and controls, p=0.049 (See Table 1.)

After adjusting for WHR, serum T-ADP levels were significantly higher in CDH sufferers (10.1±4.0 ug/ml) than in both EM (8.6±3.5 ug/ml) and controls (7.5±2.4 ug/ml), p=0.024. In addition HMW-ADP was higher in CDH (6.1±2.8 ug/ml) as compared to EM (4.2±1.7 ug/ml) and controls (3.9±1.5 ug/ml), p=0.003. MMW-ADP was also higher in CDH (2.0±1.2 ug/ml) as compared to EM (1.5±0.7 ug/ml) and controls (1.1±0.4 ug/ml), p=0.009. No significant difference was found for LMW-ADP levels or the ratio of HMW-ADP to T-ADP (HMWR), although a trend towards higher levels of LMW-ADP in CDH (2.9±2.1 ug/ml) as compared to EM (2.4±1.3 ug/ml) and controls (2.2±1.0 ug/ml, p<0.22) was seen. When groups were separated as migraine with aura and migraine without aura, no significant differences in T-ADP or any of its multimers were seen (See Table 3).

### Discussion

Adipose tissue is an important source of cytokines and adipocytokines. Adiponectin is an adipocytokine, primarily secreted from adipose tissue that has both pro and anti-inflammatory properties. Our current cross-sectional study has three significant findings in regards to the associations between migraine, adipose tissue and adiponectin. The first is that interictal serum T-ADP, HMW-ADP and MMW-ADP levels were increased in female CDH sufferers as compared with matched controls in our study. These findings support the possible pro-inflammatory role of adiponectin in the neurogenic inflammatory cascade resulting in migraine chronification.

The majority of studies evaluating ADP levels in humans have reported reduced concentrations of total adiponectin levels in metabolic disorders such as obesity and diabetes. In addition previous studies have reported that HMW-ADP and the ratio of HMW-ADP to T-ADP are better predictors of metabolic parameters and insulin sensitivity than T-ADP. However, the role of adiponectin in inflammatory and immunological diseases appears to be somewhat different than in metabolic diseases. As with our current study in CDH sufferers, elevated levels of ADP have been reported in inflammatory disorders such as arthritis, cardiovascular disease and end-stage renal disease. These studies, in conjunction with our current results, lead to our second significant finding that supports that it is the multimeric distribution of ADP which is critical in determining if ADP functions in either a pro-inflammatory or an anti-inflammatory role.

The third significant finding from our study is that the distribution of adipose tissue appears to be a significant factor in the association between adipose tissue and migraine. In our study, participants were matched based on age and sex as well as BMI, which is an estimation of total body fat (even when height and weight are directly measured as we did). We found that although total body obesity, as defined by a BMI≧30, was not significant in our participants, abdominal obesity as estimated by the WHR was significantly greater in CDH sufferers as compared to EM and controls.

The connection between abdominal obesity and migraine may be through adiponectins modulation of interleukin-6 (a proinflammatory cytokine and one of the chief hepatic inducers of C-reactive protein) as approximately 30% of circulating IL-6 is estimated to be secreted by adipocytes. Furthermore, IL-6 has been reported to be more significantly secreted from the visceral or abdominal adipose tissue; and visceral adipose tissue has been shown to be significantly associated with IL-6 concentrations in obese individuals even after adjustment for BMI.

It is interesting to note that adult females have higher T-ADP and HMW-ADP levels than males, with these levels reached at puberty. Furthermore, both T-ADP and HMW-ADP decrease through puberty in males. Similarly, prior to puberty, migraine occurs in slightly more boys than girls. However, in parallel with the puberty-related increases in T-ADP and HMW-ADP, migraine occurs in three times as many women as men following puberty. In addition, menstrually related migraine has been shown to occur most frequently two days before to three days after the onset of menses, at the time in the menstrual cycle when estrogen levels are significantly declining. As estrogen has been shown to suppress adiponectin, it would suggest that it is at this time that adiponectin may significantly rise and contribute to neurogenic inflammation through stimulation of NFkβ pathways and release of cytokines. However, testosterone modulates adiponectin levels as well. And testosterone replacement (which would cause a decrease in serum adiponectin levels) in refractory, male cluster headache sufferers, has been shown to effectively resolve headache. Thus, future studies will need to control for sex hormones in both sexes.

The present study demonstrates for the first time that T-ADP serum levels are elevated in female CDH sufferers interictally. In addition, as the individual isomers of ADP which were shown to be elevated were the HMW-ADP and MMW-ADP isomers, our data supports that HMW-ADP and MMW-ADP are the isomers responsible for the elevation of T-ADP.

It has been suggested that further evaluation of neuroendocrine peptides such as orexin may provide a link between the behavioral manifestations and the triggering of migraine. Our current work with ADP similarly falls into this category, and underscores the import of adipose tissue as a dynamic neuroendocrine organ with multiple possible links within the pain system. Further research in regards to the role of adiponectin as well as other obesity-related neuroendocrine peptides in the neuronal system is warranted.

TABLE 1

| | | Chronic Daily | | |
|---|---|---|---|---|
| | Episodic Migraine | Headache | Controls | p = value |
| N (%) | 13 (35) | 12 (32.4) | 12 (32.4) | |
| Mean Age ± SD | 34.1 ± 9.5 | 33.8 ± 9.2 | 32.5 ± 9.3 | 0.91 |
| Marital Status | | | | 0.29 |
| Single (%) | 46.2 | 50.0 | 75.0 | |
| Married (%) | 38.5 | 41.7 | 25.0 | |
| Divorced/Widowed (%) | 0.0 | 8.3 | 0.0 | |
| Mean Income | | | | 0.77 |
| <$20,000 (%) | 15.4 | 25.0 | 25.0 | |
| $20-50,000 (%) | 30.8 | 33.3 | 33.3 | |
| $50-100,000 (%) | 38.5 | 16.7 | 16.7 | |
| >$100,000 (%) | 7.7 | 16.7 | 25.0 | |
| Hx Depression (%) | 15.4 | 16.7 | 0.0 | 0.34 |
| Education | | | | 0.14 |
| <High School (%) | 0.0 | 0.0 | 0.0 | |
| High School (%) | 27.7 | 50.0 | 25.0 | |
| College (%) | 69.2 | 25.0 | 50.0 | |
| Post Graduate (%) | 23.1 | 16.7 | 25.0 | |
| Mean BMI (kg/m²) | 23.9 ± 5.1 | 24.4 ± 3.8 | 24.8 ± 5.0 | 0.93 |
| BMI ≧ 30 (%) | 8.3 | 8.3 | 16.7 | 0.56 |
| Mean WC (cm) | 75.2 ± 11.1 | 80.0 ± 13.5 | 77.8 ± 9.7 | 0.90 |
| WC = 80-88 (%) | 16.7 | 8.3 | 8.3 | 0.53 |
| WC > 88 (%) | 8.3 | 33.3 | 16.7 | 0.29 |
| Mean WHR | 0.77 ± 0.06 | 0.80 + 0.10 | 0.773 ± 0.03 | 0.66 |
| WHR > 0.85 (%) | 8.3 | 33.3 | 0.0 | 0.049 |
| Mean Glucose | 89.62 ± 11.3 | 97.25 ± 16.8 | 93.67 ± 17.8 | 0.48 |

Table 1: Demographics and Anthropometric Measurements: Demographic variables showed no significant differences. Although, no significant difference was shown in regards to those with an estimated total body fat level consistent with obesity as based on the BMI, a significant difference was found in regards to those estimated to have regional or abdominal obesity based on the WHR.

TABLE 2

| Anthropometric | | HMW- | MMW- | LMW- | |
|---|---|---|---|---|---|
| Measure | T-ADP | ADP | ADP | ADP | HMWR |
| BMI | −0.81 | −0.20 | −0.15 | −0.22 | −0.28 |
| WC | −0.19 | −0.26 | −0.15 | −0.08 | −0.22 |
| WHR | −0.38** | −0.36** | −0.15 | −0.11 | −0.09 |

Table 2: Correlation of Anthropometric measure with Adiponectin Serum Levels. Bivariate pearson correlation analysis revealed that BMI and WC was not significantly correlated with adiponectin or its multimers. However, WHR was significantly correlated both T-ADP and HMW-ADP. Values are given as a mean ±standard deviation in the table; **p-value<0.01.

TABLE 3

| | | | MMW- | LMW- | |
|---|---|---|---|---|---|
| | T-ADP | HMW-ADP | ADP | ADP | HMWR |
| (N) | (ug/ml) | (ug/ml) | (ug/ml) | (ug/ml) | (ug/ml) |
| Control (12) | 7.5 ± 2.4 | 3.9 ± 1.5 | 1.1 ± 0.4 | 2.2 ± 1.0 | 0.5 ± 0.1 |
| EM (13) | 8.6 ± 3.5 | 4.2 ± 1.7 | 1.5 ± 0.7 | 2.4 ± 1.3 | 0.5 ± 0.1 |
| CDH (12) | 10.1 ± 4.0* | 6.1 ± 2.8** | 2.0 ± 1.2** | 2.9 ± 2.1 | 0.6 ± 0.1 |
| MA (n = 12; 6 EM, 6 CDH) | 8.7 ± 3.8 | 4.5 ± 2.6 | 1.7 ± 0.74 | 2.5 ± 1.6 | 0.5 ± 0.1 |
| MO (n = 12; 7 EM, 6 CDH) | 9.9 ± 3.7 | 5.7 ± 2.3 | 1.8 ± 1.2 | 2.8 ± 1.9 | 0.6 ± 0.1 |

Table 3: Serum Levels of Total, HMW, MMW and LMW Adiponectin in Migraineurs. *p-value<0.05, **p-value<0.01; Values are given as a mean ±standard deviation.

Whereas particular embodiments of this invention have been described above for purposes of illustration, it will be evident to those skilled in the art that numerous variations of the details of the present invention may be made without departing from the invention as defined in the appended claims.

What is claimed is:

1. A method for treating a headache in a subject comprising administering a therapeutically effective amount of an adipocytokine to the subject.

2. The method of claim 1, wherein the headache is a migraine.

3. The method of claim 1, wherein the adipocytokine comprises adiponectin.

4. The method of claim 3, wherein the adiponectin comprises natural adiponectin, synthetic adiponectin, full length adiponectin, fragments of adiponectin, monomeric adiponectin, multimeric adiponectin, adiponectin agonists, adiponectin antagonists, or combinations thereof.

5. The method of claim 4, wherein the fragments of adiponectin comprise a globular head of adiponectin.

6. The method of claim 4, wherein the multimeric adiponectin comprises high molecular weight adiponectin (HMW-ADP), middle molecular weight adiponectin (MMW-ADP), low molecular weight adiponectin (LMW-ADP), or combinations thereof.

7. The method of claim 3, further comprising administering, in combination with the adiponectin, a therapeutically effective amount of an adipocytokine other than adiponectin.

8. The method of claim 7, wherein the adipocytokine comprises leptin, resistin, visfatin, adipsin, or combinations thereof.

9. The method of claim 3, further comprising administering, in combination with the adiponectin, a therapeutically effective amount of an analgesic comprising an opioid analgesic, a non-opioid analgesic, or combinations thereof.

10. The method of claim 9, wherein the non-opioid analgesic comprises a non-steroidal anti-inflammatory agent.

11. The method of claim 10, wherein the non-steroidal anti-inflammatory agent comprises propionic acid derivatives, acetic acid derivatives, fenamic acid derivatives, biphenylcarboxylic acid derivatives, salicylates, pyrazolones, oxicams, salicylic acid derivatives, or combinations thereof.

12. The method of claim 9, wherein the opioid analgesic comprises alfentanil, apomorphine, buprenorphine, butorphanol, codeine, dextropropoxyphene, diamorphine, dihydrocodeine, fentanyl, hydrocodone, hydromorphone, levorphanol, meperidine, meptazinol, methadone, morphine, nalbuphine, oxycodone, oxymorphone, pentazocine, propoxyphene, sufentanil, tramadol, or combinations thereof.

13. The method of claim 3, further comprising administering, in combination with the adiponectin, a therapeutically effective amount of an orexin modulating agent.

14. The method of claim 3, further comprising administering, in combination with the adiponectin, a therapeutically effective amount of a dopamine modulating agent.

15. The method of claim 14, wherein the dopamine modulating agent comprises metoclopramide, prochlorpromaxine, or combinations thereof.

16. The method of claim 3, further comprising administering, in combination with the adiponectin, a therapeutically effective amount of a serotonin modulating agent.

17. The method of claim 16, wherein the serotonin modulating agent comprises triptans, SNRI compounds, SSRI compounds, or combinations thereof.

18. The method of claim 3, further comprising administering, in combination with the adiponectin, a therapeutically effective amount of an anti-nausea agent.

19. The method of claim 18, wherein the anti-nausea agent comprises metoclopramide, prochlorperazine, osansetrom, or combinations thereof.

20. The method of claim 3, wherein the adiponectin is administered in a pharmaceutical composition.

21. The method of claim 20, wherein the pharmaceutical composition comprises adiponectin and a pharmaceutically acceptable carrier or excipient.

22. The method of claim 21, wherein the pharmaceutically acceptable carrier or excipient comprises water, salt solutions, saline, buffered saline, alcohols, glycerol, ethanol, gum arabic, vegetable oils, benzyl alcohols, polyethylene glycols, gelatin, carbohydrates, lactose, amylase, starch, dextrose, magnesium stearate, talc, silicic acid, viscous paraffin, perfume oil, fatty acid esters, hydroxymethylcellulose, polyvinyl pyrolidone, or combinations thereof.

23. The method of claim 20, wherein the pharmaceutical composition further comprises a therapeutically effective amount of an adipocytokine other than adiponectin.

24. The method of claim 23, wherein the adipocytokine comprises leptin, resistin, visfatin, adipsin, or combinations thereof.

25. The method of claim 20, wherein the pharmaceutical composition further comprises a therapeutically effective amount of an analgesic comprising an opioid analgesic, a non-opioid analgesic, or combinations thereof.

26. The method of claim 25, wherein the non-opioid analgesic comprises a non-steroidal anti-inflammatory agent.

27. The method of claim 26, wherein the non-steroidal anti-inflammatory agent comprises propionic acid derivatives, acetic acid derivatives, fenamic acid derivatives, biphenylcarboxylic acid derivatives, salicylates, pyrazolones, oxicams, salicylic acid derivatives, or combinations thereof.

28. The method of claim 25, wherein the opioid analgesic comprises alfentanil, apomorphine, buprenorphine, butorphanol, codeine, dextropropoxyphene, diamorphine, dihydrocodeine, fentanyl, hydrocodone, hydromorphone, levorphanol, meperidine, meptazinol, methadone, morphine, nalbuphine, oxycodone, oxymorphone, pentazocine, propoxyphene, sufentanil, tramadol, or combinations thereof.

29. The method of claim 20, wherein the pharmaceutical composition further comprises a therapeutically effective amount of an orexin modulating agent.

30. The method of claim 20, wherein the pharmaceutical composition further comprises a therapeutically effective amount of a dopamine modulating agent.

31. The method of claim 30, wherein the dopamine modulating agent comprises metoclopramide, prochlorpromaxine, or combinations thereof.

32. The method of claim 20, wherein the pharmaceutical composition further comprises a therapeutically effective amount of a serotonin modulating agent.

33. The method of claim 32, wherein the serotonin modulating agent comprises triptans, SNRI compounds, SSRI compounds, or combinations thereof.

34. The method of claim 20, wherein the pharmaceutical composition further comprises a therapeutically effective amount of an anti-nausea agent.

35. The method of claim 34, wherein the anti-nausea agent comprises metoclopramide, prochlorperazine, osansetrom, or combinations thereof.

36. The method of claim 20, wherein the pharmaceutical composition is prepared for oral, enteral, parenteral, pulmonary, nasal, subcutaneous, intramuscular, intravenous, transdermal, topical, suppository, transmucosal, subdermal delivery, or combinations thereof.

37. The method of claim 3, wherein the therapeutically effective amount of adiponectin comprises a total daily dosage amount of 0.01 μg/ml to 100 μg/ml.

38. The method of claim 37, wherein the therapeutically effective amount of an adiponectin comprises a total daily dosage amount of 1 μg/ml to 25 μg/ml.

39. The method of claim 37, wherein the total daily dosage amount is administered once a day.

40. The method of claim 37, wherein the total daily dosage amount is administered more than once a day.

41. The method of claim 20, wherein the pharmaceutical composition is a controlled or sustained release formulation.

42. A pharmaceutical composition comprising adipocytokine and a pharmaceutically acceptable carrier or excipient for administration of a therapeutically effective amount of the adipocytokine to treat a subject with a headache.

43. The pharmaceutical composition of claim 42, wherein the headache is a migraine.

44. The pharmaceutical composition of claim 42, wherein the adipocytokine comprises adiponectin.

45. The pharmaceutical composition of claim 44, wherein the adiponectin comprises natural adiponectin, synthetic adiponectin, full length adiponectin, fragments of adiponectin, monomeric adiponectin, multimeric adiponectin, adiponectin agonists, adiponectin antagonists, or combinations thereof.

46. The pharmaceutical composition of claim 45, wherein the fragments of adiponectin comprise a globular head of adiponectin.

47. The pharmaceutical composition of claim 45, wherein the multimeric adiponectin comprises high molecular weight adiponectin (HMW-ADP), middle molecular weight adiponectin (MMW-ADP), low molecular weight adiponectin (LMW-ADP), or combinations thereof.

48. The pharmaceutical composition of claim 44, further comprising an adipocytokine other than adiponectin.

49. The pharmaceutical composition of claim 48, wherein the adipocytokine comprises leptin, resistin, visfatin, adipsin, or combinations thereof.

50. The pharmaceutical composition of claim 44, further comprising an analgesic comprising an opioid analgesic, a non-opioid analgesic, or combinations thereof.

51. The pharmaceutical composition of claim 50, wherein the non-opioid analgesic comprises a non-steroidal anti-inflammatory agent.

52. The pharmaceutical composition of claim 51, wherein the non-steroidal anti-inflammatory agent comprises propionic acid derivatives, acetic acid derivatives, fenamic acid derivatives, biphenylcarboxylic acid derivatives, salicylates, pyrazolones, oxicams, salicylic acid derivatives, or combinations thereof.

53. The pharmaceutical composition of claim 50, wherein the opioid analgesic comprises alfentanil, apomorphine, buprenorphine, butorphanol, codeine, dextropropoxyphene, diamorphine, dihydrocodeine, fentanyl, hydrocodone, hydromorphone, levorphanol, meperidine, meptazinol, methadone, morphine, nalbuphine, oxycodone, oxymorphone, pentazocine, propoxyphene, sufentanil, tramadol, or combinations thereof.

54. The pharmaceutical composition of claim 44, further comprising an orexin modulating agent.

55. The pharmaceutical composition of claim 44, further comprising a dopamine modulating agent.

56. The pharmaceutical composition of claim 55, wherein the dopamine modulating agent comprises metoclopramide, prochlorpromaxine, or combinations thereof.

57. The pharmaceutical composition of claim 44, further comprising a therapeutically effective amount of a serotonin modulating agent.

58. The pharmaceutical composition of claim 57, wherein the serotonin modulating agent comprises triptans, SNRI compounds, SSRI compounds, or combinations thereof.

59. The pharmaceutical composition of claim 44, further comprising a therapeutically effective amount of an anti-nausea agent.

60. The pharmaceutical composition of claim 59, wherein the anti-nausea agent comprises metoclopramide, prochlorperazine, osansetrom, or combinations thereof.

61. The pharmaceutical composition of claim 44 prepared for oral, enteral, parenteral, pulmonary, nasal, subcutaneous, intramuscular, intravenous, transdermal, topical, suppository, transmucosal, subdermal delivery, or combinations thereof.

62. The pharmaceutical composition of claim 44, wherein the adiponectin is present in the pharmaceutical composition at an amount of 0.01 μg/ml to 100 μg/ml.

63. The pharmaceutical composition of claim 62, wherein the adiponectin is present in the pharmaceutical composition at an amount of 5 μg/ml to 25 μg/ml.

64. The pharmaceutical composition of claim 44, wherein the pharmaceutical composition is administered once a day.

65. The pharmaceutical composition of claim 44, wherein the pharmaceutical composition is administered more than once a day.

66. The pharmaceutical composition of claim 44, wherein the pharmaceutical composition is a controlled or sustained release formulation.

67. The pharmaceutical composition of claim 44, wherein the pharmaceutically acceptable carrier or excipient comprises water, salt solutions, saline, buffered saline, alcohols, glycerol, ethanol, gum arabic, vegetable oils, benzyl alcohols, polyethylene glycols, gelatin, carbohydrates, lactose, amylase, starch, dextrose, magnesium stearate, talc, silicic acid, viscous paraffin, perfume oil, fatty acid esters, hydroxymethylcellulose, polyvinyl pyrolidone, or combinations thereof.

68. Use of adipocytokine for manufacture of a medicament for treatment of a headache.

69. The use of claim 68, wherein the headache is a migraine.

70. The use of claim 68, wherein the adipocytokine comprises adiponectin.

71. A method of diagnosing the presence or absence of a headache in a subject, the method comprising: assessing a test sample from the subject for the level of adiponectin, wherein the presence of a level of adiponectin that is greater than a level of adiponectin in a comparable negative control sample, by an amount that is statistically significant, is indicative of the presence of headache in the subject, and wherein the presence of a level of adiponectin that is equal to or less than a level of adiponectin in a comparable negative control sample, by an amount this is statistically significant, is indicative of the absence of headache in the subject.

72. The method of claim 71, wherein the headache is a migraine.
